(19) United States
(12) Patent Application Publication
(10) Pub. No.: US 2008/0118537 A1
(43) Pub. Date: May 22, 2008

(54) Cosmetic composition containing hydrophilic spherical polymethylsilsesquioxane powder

(75) Inventors: James J. Wang, Albertson, NY (US); Jin L. Hung, Fairview, NJ (US)

Correspondence Address: K.F. ROSS P.C., 5683 RIVERDALE AVENUE, SUITE 203 BOX 900, BRONX, NY, 10471-0900, US

(73) Assignee: GRANT INDUSTRIES, INC.

(21) Appl. No.: 11/603,269

(22) Filed: Nov. 21, 2006

(51) Int. Cl. A61K 8/84; A61Q 19/00

(52) U.S. Cl. 424/401; 523/105; 424/78.03

(57) ABSTRACT

A cosmetic composition is disclosed containing hydrophilic, spherical powder of polymethylsilsesquioxane having a particle size ranged from 1-20 microns with a narrow particle size distribution. The hydrophilic, spherical powder in the composition is prepared by a process comprising (1) hydrolyzing and condensing methyltrimethoxysilane in water in the presence of a thickener as suspending agent, an oil as surface tension modifier and an acid or base as catalyst to form spherical particles, (2) post-hydrolyzing the spherical particles in an aqueous NaOH solution and (3) purifying the spherical particles by repeated washing and vacuum stripping.

### FIELD OF THE INVENTION

The present invention relates to a hydrophilic spherical powder of polymethylsilsesquioxane (PMSQ) and to a process for the preparation thereof. This invention relates further to a hydrophilic cosmetic composition containing the hydrophilic spherical powder of PMSQ. The invention relates further to a hydrophilic cosmetic composition containing the hydrophilic spherical powder of PMSQ that is oil-free and surfactant-free, such as moisturizers, aqueous liquid foundations, and aqueous UV protection lotions.

### BACKGROUND OF THE INVENTION

Polymethylsilsesquioxane (PMSQ) powders, especially spherical powders, are frequently used in cosmetic formulations to obtain the benefits of excellent skin sensory feel, light diffusing effect, smooth texture, anti-caking and water repellency. In comparison with other synthetic polymer powders, PMSQ powders have an excellent heat resistance up to 400° C. and a higher purity because the residual byproducts and monomers can be easily removed by drying at about 300° C., at which temperature most polymer powders are decomposed or discolored. Many methods have been proposed in the art for the preparation of PMSQ powders. Belgian Patent 572,412 disclosed a process in which ethyltrichlorosilane is hydrolytically condensed in water. U.S. Pat. No. 4,528,390 in 1985 discloses a process to make spherical PMSQ powder in which methyltrimethoxysilane or partially hydrolyzed condensate was hydrolyzed and condensed in an aqueous solution of ammonia followed by washing, drying and pulverizing. According to that invention, the resulting powder is very hydrophobic and compatible with oils, and is useful as an additive ingredient for cosmetic applications. Such spherical PMSQ powder disclosed above is currently available from GE Toshiba under the trade name of Tospearl 2000 and 145A. U.S. Pat. No. 4,892,726 disclosed a cosmetic composition containing this powder. U.S. Pat. No. 4,871,616 disclosed a process to modify chemically the surface of spherical PMSQ powder with metal alkoxide and alkoxysilane with an amino radical, resulting in a positive-charged powder. Due to the hydrophobic nature, the above powders cannot be directly incorporated in an aqueous solution or suspension without using a surfactant, which is one of the major sources for skin irritation. Further, the above powders are barely compatible with glycerin and glycols. Therefore, it is difficult to be used in some aqua formulations such as skin toner, aqua moisturizing lotion, glycerin hand lotion, etc. It remains desirable to obtain a hydrophilic PMSQ powder substantially compatible with water, glycerin and glycols.

### OBJECTS OF THE INVENTION

It is a first object of this invention to provide a spherical, hydrophilic PMSQ powder having a particle size of 1-20 microns with a narrow particle size distribution fulfilling the benefits resulting from the use of hydrophilic, spherical PMSQ powder.

It is a second object of this invention to provide a cosmetic hydrophilic PMSQ powder, substantially compatible with water, glycerin and glycols, that is useful for making oil-free, surfactant-free cosmetic products with a very smooth feel.

It is a third object of this invention to provide a stable skin treatment composition that provides a smooth feel with a matte appearance.

It is a fourth object of this invention to provide a stable make-up composition that provides a unique feel on the skin and a natural color.

### DETAILED DESCRIPTION OF THE INVENTION

The present invention is directed to a hydrophilic, spherical PMSQ powder having a particle size of 1-20 microns with a narrow particle size distribution. One of the most characteristic features of the powder is the hydrophilic property that is attributed to the hydroxyl group on PMSQ backbone, which make the powder absorb and hold as much as 55% of water relative to its own weight. The powder can be directly mixed with water or glycerin or glycols to provide an excellent slipperiness and moisturizing feeling on the skin.

Unless otherwise indicated, all percentages and ratios are by weight and all weight percentages are calculated on the basis of the total weight of the composition.

A preferred narrow particle size distribution for the spherical, hydrophilic PMSQ powder is as follows: 99% or more of the particles are within 1 to 20 microns and 70% or more of the particles are within ±30% of the mean value of the particle size.

The spherical, hydrophilic PMSQ powder is prepared by a process comprising the steps of (1) hydrolyzing and condensing methyltrimethoxysilane in water in the presence of a thickener as suspending agent, an oil as surface tension modifier and an acid or base as catalyst to form spherical particles, (2) post-hydrolyzing the spherical particles in an aqueous NaOH solution and (3) purifying the spherical particles by repeated washing and vacuum stripping.

In the first step, methyltrimethoxysilane is preferred to be the starting material due to its economic cost. Most commercial methyltrimethoxysilane with a purity of 98% or higher undergoes hydrolysis upon contacting with moisture or water at room temperature. The hydrolysis is not an equilibrium reaction because some insoluble products are yielded and precipitated. In the initial stage of the reaction, soluble intermediate methoxy derivatives of methylsilsesquioxane or polysilicates form and grow in molecular weight until most of the methoxy groups are removed, resulting in a milky white suspension of particles having a non-linear network structure of (MeSiO_{1.5}) with residual methoxy groups and a substantial amount of un-condensed hydroxyl groups due to stereo hindrance. Dilute HCl and acetic acid can be used to promote the hydrolysis reaction and facilitate the formation of the condensation product, while aqueous ammonia or triethanolamine can be used to facilitate a more complete condensation. When the hydrolysis occurs at moderate acidic condition, the particle size tends to be small. When at a strong basic condition, the particle size tends to be large because the coalescence of oligo-polymer droplets is more likely to occur. In general, the particle size and distribution are determined by a few factors such as the catalyst used and its concentration, reaction temperature, mixing speed, interface tension and the feeding ratio of monomer/water. As the reaction is exothermic, a large amount of water is necessary to absorb the released heat to control the reaction temperature. An oil can be used to alter the surface tension between the oil phase and the water phase, where the reaction occurs to form spherical particles. The oil can be selected from silicone oil, mineral oil or synthetic oil. Mineral oil or silicone oil, especially low viscosity silicone oil is preferred. A water-soluble thickener is also found useful as a suspending agent to prevent the full-grown particles from coagulating with each other and warrant a narrow particle size distribution. The thickener could be selected from starch, xanthan gum, hydroxyethylcellulose, etc.

In the second step, the particles separated from the suspension are treated with 0.5 to 1% of NaOH relative to the total weight of the batch over 24 hours at room temperature. This treatment allows for a further hydration of residual methoxy groups on the network backbone and an ionization of polysilicic acid as the side-reaction product.

In the third step, repeated washing-soaking cycles with a large amount of water followed by soaking-vacuum stripping cycles are used to remove the residual monomer and the byproducts of methanol and polysilicates. The product is recovered through filtration as a wet powder containing typically 42 to 54% water relative to its own weight. The water content of the powder can be varied by partially drying under reduced pressure.

The hydrophilic PMSQ powder is uniquely useful for formulating oil-free, surfactant-free cosmetic compositions due to its substantial compatibility with water. The cosmetic compositions of the present invention are prepared by combining the hydrophilic PMSQ powder with water, glycerin, glycols, active ingredients, preservatives, and optionally other ingredients such as oil, wax, dye, surfactant, thickener, UV agent, pigments, powders, fragrance, etc.

Mention may be made of any additives usually delivered to the skin, such as fillers and/or pearlescent agents, antifoam agents, antioxidants, opacifiers, fragrances, preserving agents, cosmetic or pharmaceutical active agents, sunscreens, antiperspirant agents and self-tanning agents, each in an effective amount to accomplish its respective functions. A person skilled in the art may select these possible additional compounds in such an amount that the advantageous properties of the composition according to the present invention are not, or are substantially not, adversely affected by the addition envisaged. The compositions are not particularly restricted to any format of, for example, gel, lotion, cream, foundation, loose powder, press powder, stick, soap and paste.

The invention is illustrated in the examples below, which are not intended to be restrictive. Examples 1 to 3 describe the preparation of hydrophilic PMSQ powder used in the present invention. Examples 4 and 5 are comparative examples comparing the physical properties of cosmetic compositions containing the spherical, hydrophilic PMSQ powder according to is the present invention with the physical properties of prior art cosmetic compositions containing the spherical, hydrophobic PMSQ powders known as Tospearl 2000 according to the prior art. Examples 6 to 8 describe the topical cosmetic end product compositions of the present invention containing the spherical, hydrophilic PMSQ powder. All parts and percentages referred to herein are by weight relative to the total weight of the composition and the viscosity referred to herein is at 25° C. unless otherwise indicated.

### EXAMPLES

### Example 1

Preparation of Hydrophilic PMSQ Powder Under Acidic Condition

To a reactor containing 100 parts of water and 0.02 part of xanthan gum at 15° C. were added 28 parts of methyltrimethoxysilane and 2 parts of cyclopentasiloxane. The mixture was mixed very slowly at 5 rpm at 25-29° C. and pH-3.4 for 24 hours. A particulate product partially precipitated to the bottom in the reactor was collected by centrifuging and added to a strong base solution of 20 parts of water and 0.4 part of NaOH to allow for a post-hydration reaction at room temperature for 24 hours. The product was washed at least three time or until the pH was neutral by using water followed by centrifuging. The residual methanol was generally about 0.05% at this stage. A vacuum stripping was used to remove the methanol until its level was below 80 ppm as determined from a gas chromatography spectrum. The resulting particles had a spherical shape and the particle size was between 1-4 microns with a mean value of 2.5 microns. The product was a wet, flowable powder.

### Example 2

Preparation of Hydrophilic PMSQ Powder Under Basic Condition

To a reactor containing 100 parts of water, 0.06 part of xanthan gum and 0.07 part of 28% aqueous ammonia at 15° C. were added 28 parts of methyltrimethoxysilane and 3 parts of isohexadecane. The mixture was mixed at 300 rpm at 15-22° C. and pH 9.5-10.0 for 3 hours and kept in the reactor for 24 hours without mixing, yielding a particulate product that was precipitated to the bottom in the reactor. The rest of the procedures were essentially the same as described in Example 1. The resulting particles had a spherical shape and the particle size was between 5-13 microns with a mean value of 10 microns. The product was a wet, flowable powder.

### Example 3

Preparation of Hydrophilic PMSQ Powder Under Basic Condition

To a reactor containing 100 parts of water, 0.12 part of hydroxyethylcellulose and 0.18 part of 28% aqueous ammonia at 15° C. were added 28 parts of methyltrimethoxysilane and 3 parts of isohexadecane. The mixture was mixed at 5 rpm at 15-28° C. and pH 9.5-10.0 for 3 hours and kept in the reactor for 24 hours without mixing, yielding a particulate product that was precipitated to the bottom in the reactor. The rest of the procedures were essentially the same as described in Example 1. The resulting particles had a spherical shape and the particle size was between 1-4 microns with a mean value of 2 microns. The product was a wet, flowable powder.

### Example 4

Comparative Cosmetic Composition: Glycerin Lotion

| | Formula 4.1 | Formula 4.2 |
|---|---|---|
| PMSQ Powder of | 30.00 | 0 |
| Example 2 | | |
| Tospearl 2000 | 0 | 30.00 |
| De-ionized Water | 49.50 | 49.50 |
| Glycerin | 20.00 | 20.00 |
| Preservative | 0.50 | 0.50 |
| Total | 100.00 | 100.00 |

Formula 4.1 is a uniform, translucent lotion and has a smooth, non-greasy feel. Formula 4.2 shows a separation of Tospearl 2000 powder on top of the liquid.

### Example 5

Comparative Cosmetic Composition: Oil-Free and Surfactant-Free Moisturizing Serum

| | Formula 5.1 | Formula 5.2 |
|---|---|---|
| PMSQ Powder of | 25.00 | 0 |
| Example 2 | | |
| Tospearl 2000 | 0 | 25.00 |
| Glycerin | 24.00 | 24.00 |
| Pentylene Glycol | 3.00 | 3.00 |
| 1,3-Butylene Glycol | 2.00 | 2.00 |
| Allantoin | 0.10 | 0.10 |
| Tocopheryl Acetate | 0.25 | 0.25 |
| De-ionized Water | 39.65 | 39.65 |
| Ascorbyl Glucoside | 1.00 | 1.00 |
| Niacinamide | 2.00 | 2.00 |
| 1% Sodium | 3.00 | 3.00 |
| Hyaluronate | | |
| Total | 100.00 | 100.00 |

Formula 5.1 is a uniform, translucent serum and provides a smooth, moisturizing feel. Formula 5.2 shows a separation of Tospearl 2000 powder on top of the liquid.

### Example 6

Cosmetic Composition: Moisturizing Pressed Powder

| | | |
|---|---|---|
| (1) | Mica | 46.60 |
| | PMSQ Powder of Example 2 | 20.00 |
| | Nylon 12 | 5.00 |
| | Ultragel 300 | 1.00 |
| | De-ionized water | 4.00 |
| | Methylparaben | 0.20 |
| | Iron Oxide-Yellow | 1.10 |
| | Iron Oxide-Red | 0.20 |
| | Iron Oxide-Black | 0.10 |
| | TiO2 | 5.00 |
| | Mica | 5.00 |
| (2) | Isononyl Isononanoate | 6.00 |
| | Dimethicone, 10 cSt | 1.80 |
| | Squalane | 1.50 |
| | Phenoxyethanol | 0.50 |
| | | 100.00 |

### The formula provides a very slippery, moisturizing feel.

### Example 7

Cosmetic Composition: Aqueous Liquid Foundation

| | |
|---|---|
| De-ionized Water | 49.20 |
| PMSQ Powder of Example 2 | 15.00 |
| Polyacryloyldimethyl Taurate | 1.00 |
| Glycerin | 15.00 |
| 1,3 Butylene Glycol | 4.00 |
| Pentylene Glycol | 3.00 |
| Phenoxyethanol | 0.80 |
| Iron Oxide Yellow | 1.05 |
| Iron Oxide Red | 0.32 |
| Iron Oxide Black | 0.11 |
| RBTD-MS2, TiO2 | 5.26 |
| Mica | 5.26 |
| Total | 100.00 |

### Example 8

Cosmetic Composition: Aqueous UV Protection Lotion, SPF 26

| | |
|---|---|
| De-ionized Water | 26.00 |
| PMSQ Powder of Example 2 | 30.00 |
| Glycerin | 15.00 |
| Ultra Fine Zinc Oxide | 20.00 |
| Pentylene Glycol | 3.00 |
| 1,3 Butylene Glycol | 3.00 |
| 1% Sodium Hyaluronate | 1.50 |
| Polyacryloyldimethyl Taurate | 1.00 |
| Phenoxyethanol | 0.50 |
| | 100.00 |

We claim:

1. A hydrophilic, spherical powder of polymethylsilsesquioxane having a particle size range from 1-20 microns with a narrow particle size distribution.

2. The hydrophilic, spherical powder defined in claim 1, prepared by a process comprising (a) hydrolyzing and condensing trialkoxysilane in water in the presence of a thickener as suspending agent, an oil as surface tension modifier and an acid or base as catalyst to form spherical particles, (b) post-hydrolyzing the spherical particles in an aqueous NaOH solution and (c) purifying the spherical particles by repeated washing and vacuum stripping.

3. The hydrophilic, spherical powder of polymethylsilsesquioxane prepared according to claim 2, wherein the trialkoxysilane is methyltrimethoxysilane.

4. The hydrophilic, spherical powder of polymethylsilsesquioxane prepared according to claim 2, wherein the thickener as suspending agent is a water soluble compound.

5. The hydrophilic, spherical powder of polymethylsilsesquioxane prepared according to claim 2, wherein the oil is a low viscosity silicone fluid or hydrocarbon fluid.

6. The hydrophilic, spherical powder of polymethylsilsesquioxane prepared according to claim 2, wherein the acid is acetic acid and the base is aqueous ammonia.

7. The hydrophilic, spherical powder of polymethylsilsesquioxane prepared according to claim 2, containing 0.5 to 45% of water of hydration relative to its own weight.

8. The hydrophilic, spherical powder of polymethylsilsesquioxane prepared according to claim 4 wherein the water soluble thickener is xanthan gum or hydroxyethylcellulose.

9. The hydrophilic, spherical powder of polymethylsilsesquioxane defined in claim 1, wherein the narrow particle size distribution of the polymethylsilsesquioxane powder is as follows: 99% or more of the particles are within 1 to 20 microns and 70% or more of the particles are within ±30% of the mean value of the particle size.

10. A cosmetic composition comprising:
0.5% to 65% by weight of a hydrophilic, spherical polymethylsilsesquioxane powder having a particle size range of 1 to 20 microns with a narrow particle size distribution; and which further comprises at least one of the following ingredients:
(a) 1 to 99.5% of an aqueous solution that may contain glycerin, glycols and aqueous thickening agents;
(b) 1 to 95% of a volatile cosmetic fluid;
(c) 0.5 to 90% of a silicone fluid with viscosity 1 range of 0.5 to 350 cSt;
(d) 0.1 to 80% of an oil chosen from plant or synthetic oils, waxes, polymers or the mixture thereof;
(e) 0.05 to 15% of a surface active agent;
(f) 1 to 50% of UV agents;
(g) 0.05 to 40% of cosmetic pigments; and
(h) 0.1 to 40% of herbal extracts medicaments.

11. The cosmetic composition in claim 10 further comprising other ingredients deliverable to the skin.

12. The cosmetic composition in claim 10 comprising no oils and no surfactants.

13. A process for preparing a hydrophilic, spherical powder of polymethylsilsesquioxane having a particle size of 1-20 microns with a narrow particle size distribution, which comprises the steps of:
(a) hydrolyzing and condensing trialkoxysilane in water in the presence of a thickener as suspending agent, an oil as surface tension modifier and an acid or base as catalyst to form spherical particles;
(b) post-hydrolyzing the spherical particles in an aqueous NaOH solution and
(c) purifying the spherical particles by repeated washing and vacuum stripping.

14. The hydrophilic, spherical-powder of polymethylsilsesquioxane having a particle size range from 1-20 microns with a narrow particle size distribution prepared by the process defined in claim 13.
